(19) United States
(12) Patent Application Publication
(10) Pub. No.: US 2007/0078407 A1
(43) Pub. Date: Apr. 5, 2007

(54) Safety syringe

(76) Inventor: Hung-Chi Huang, Taichung City (TW)

Correspondence Address: Hung-Chi Huang, 235 Chung - Ho, Box 8-24, Taipei, TW

(21) Appl. No.: 11/204,932

(22) Filed: Aug. 17, 2005

(51) Int. Cl. A61M 5/32; A61M 5/00

(52) U.S. Cl. 604/198; 604/110

(57) ABSTRACT

A safety syringe comprises a securing mount, made of a flexible rubber material, having an outer wall and an inner wall. The inner wall of the safety syringe further includes a first securing part, a second securing part and a needle unit. The first securing part will be engaged by a hooked head atop a plunger when the syringe is used and ready for being dumped, whereby the securing part and the needle unit will be pulled back into a receptacle within a syringe barrel.

### FIELD OF THE INVENTION

The present invention relates to safety syringes, more particularly to a safety syringe the structure of which is simple and has precision sizes. The safety syringe is expendable.

### BACKGROUND OF THE INVENTION

A safety syringe of the prior art comprises a barrel and an enclosed part, which are made of a single material. The barrel further includes an outer sleeve made of a hard plastic material and an inner plunger made of a rubber material, whereby a hooked head extended from the plunger will engage with the enclosed part at the front end of the sleeve, when the plunder is inserted onto the sleeve. Therefore, a needle unit of the syringe will be pulled back by the plunder as an injection is completed, preventing the syringe from being used again.

Although the manufacture of the safety syringe of the prior art is simple because of its structural simplicity, it still has the following disadvantages.

1. The outer sleeve, made of a hard material, has to be made by a precision machine, so that the engagement of the sleeve and the inner plunger is tight enough to prevent the solution for injection from a leakage. The connection cannot also be too tight to smoothly pull back the plunger.

2. In spite of precision machining, the connection between the outer barrel and the inner plunger cannot be tight due to the difference between the properties of the materials making the barrel and the plunger, possibly leading to a leakage of medical solution.

3. The manufacturing process is still too complicated. First, the barrel is formed by injection molding and is put in another mold for the injection molding of the plunger.

### SUMMARY OF THE INVENTION

The primary objective of the present invention is to provide a safety syringe having a simple securing mount for mounting a needle unit, whereby the manufacturing process will be simple too.

The secondary objective of the present invention is to provide a safety syringe wherein the connection among the securing mount, a barrel, the needle unit and a plunger is stable.

The present invention provides a safety syringe. The safety syringe comprises a barrel being a hollow cylinder and having a tapered section at a front end thereof, said tapered section further including a longitudinal through hole of smaller diameter than a inner diameter of the inner receptacle defined by said barrel, a stopper being formed between said receptacle and said through hole; a securing mount, made of a soft rubber material, and further including an inner wall and a outer wall located within said through hole of said tapered section, said securing mount further having a first securing part extended inwardly from said inner wall close to said receptacle and an enlarged disc portion, whereby said stopper of said barrel will retain said securing mount; needle unit situating at a free end opposite to said retained end where said first securing part is formed, said needle unit further including a needle mount and a needle inserted in said needle mount; and a plunger inserted along said barrel and capable of being slid within said barrel, said plunger further including a push rod and a piston mounted on said push rod, said piston being provided with a hooked head that can be engaged with said first securing part as sad plunger is pushed forward deeply.

The various objects and advantages of the present invention will be more readily understood from the following detailed description when read in conjunction with the appended drawings.

### BRIEF DESCRIPTION OF THE DRAWINGS

FIG. 1 is an exploded perspective view of a safety syringe according to the present invention.

FIG. 2 is a lateral cross-sectional view of the safety syringe in FIG. 1, wherein the plunger is out of the securing mount.

FIG. 3 is a lateral cross-sectional view of the safety syringe in FIG. 1, wherein the plunger is inserted into the securing mount.

FIG. 4 is a lateral cross-sectional view of the safety syringe in FIG. 1, wherein the plunger is pulled back.

FIG. 5 is a lateral cross-sectional view of another preferred embodiment of the present invention as a safety syringe.

FIG. 6 is a lateral cross-sectional view of the safety syringe in FIG. 5, wherein the needle unit is pulled back into the barrel.

FIG. 7 is a lateral cross-sectional view of the second preferred embodiment of the present invention as a safety syringe.

FIG. 8 is a local cross-sectional view of the securing mount of the safety syringe in FIG. 7.

FIG. 9 is a lateral cross-sectional view of the third preferred embodiment of the present invention as a safety syringe.

FIG. 10 is a local cross-sectional view of the securing mount and the needle unit of the third preferred embodiment of the present invention as a safety syringe.

### DETAILED DESCRIPTION OF THE PREFERRED EMBODIMENTS

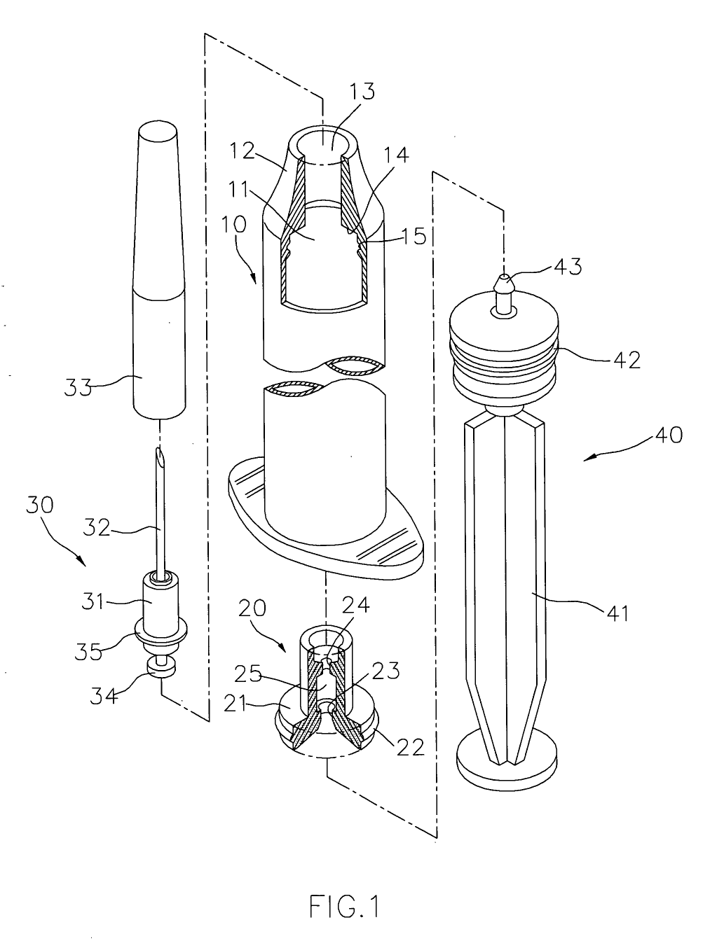

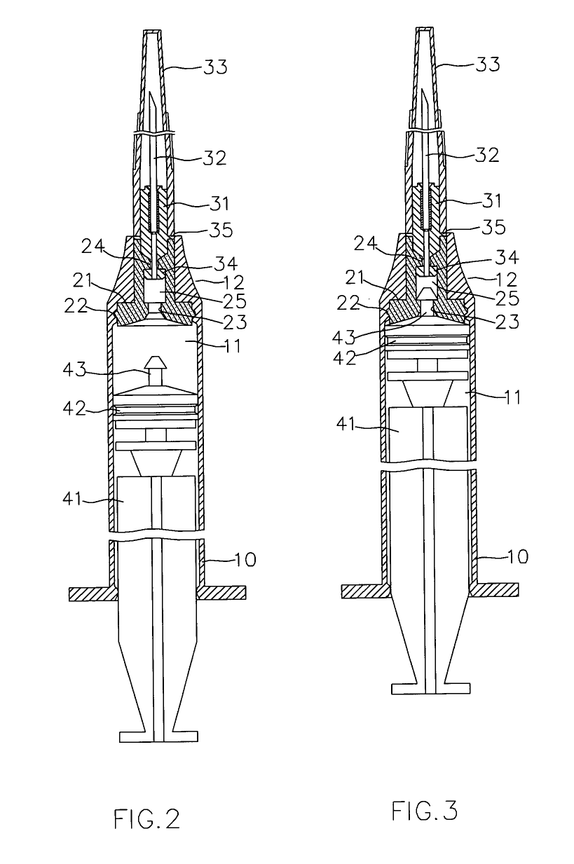

Referring to FIG. 1, a safety syringe according to the present invention comprises a barrel 10, a securing mount 20 installed within the barrel 10, a needle unit 30 mounted onto the securing mount 20 and a plunger 40 silidably inserted along the axis of the barrel 10.

Being a hollow cylinder, the barrel 10 is provided with a receptacle 11, a tapered portion 12 at the front end of the barrel 10 and a through hole 13 connecting the receptacle 11 and the tapered portion 12. There are a stopper 14 and an annular groove 15 formed between the through hole 13 and the receptacle 11.

The securing mount 20 is an inverse T shaped tapered hollow body comprises an inner wall and an outer wall. The outer wall is made of a flexible rubber material. The securing mount 20 is attached to the through hole 13 within the tapered portion 12, thereby the outer wall of the securing mount 20 and the inner wall of the through hole 13 being tightly contacted, Further, the lower terminal of the securing mount 20 is supported against the disc portion 21 of the stopper 14, and an annular projection 22 and the annular groove 15 are engaged so that the securing mount 20 will have an enlarged disc portion 21 for preventing a departure of the securing mount 20 driven by excessive pressure of the medical solution.

Further, the inner wall of the securing mount 20 close to the receptacle 11 is provided with an inwardly and transversely extended first securing part 23 and a second securing part 24 extended away from the receptacle 11. There is a receptacle section 25 between the first securing part 23 and the second securing part 24, the first securing part 23 and the second securing part 24 are connected by a axial through hole for the passage of the needle unit 30 and the plunger 40, whereby they can be retained by the first securing part 23 and the second securing part 24 respectively.

The needle unit 30 has a needle base 31, a needle 32 mounted on the needle base 31 and a needle cap 33 covering the exterior of the needle 32. The needle base 31 has an insertion through hole 13 which passes the second securing part 24 and is connected to an engagement head 34 within the receptacle section 25. Further, there is a retaining ring 35 located at a shoulder section in the second securing part 24 for retaining the needle cap 33.

The plunger 40 is slidably inserted into the receptacle 11 of the barrel 10 and comprises a rod 41, a piston 42 and a hooked head 43. The rod 41 can slide along the barrel 10; the piston 42 is mounted on one end of the rod 41 and has the hooked head 43 extended at one end thereof. The hooked head 43 is flexible and can thus go through the through hole of the first securing part 23. After passing the through hole, the largest outer diameter of the hooked head 43 is slightly larger than the inner diameter of the through hole, thereby being secured with the securing mount 20 stably.

To assemble the safety syringe, the connecting part at the bottom end of the needle unit 30 goes through the through hole of the second securing part 24 and is connected with the inner shoulder portion of the second securing part 24. Then the securing mount 20 is placed into the through hole 13 of the tapered portion 12, going through the disc portion 21 and the annular projection 22, and is engaged with the stopper 14 and the annular groove 15, whereby the securing mount 20 will not fall off. Eventually, the plunger 40 is inserted into the receptacle 11 to complete the assembly.

Referring to FIG. 3, after the safety syringe is used, a user pushes the plunger 40 deep into the barrel 10 so as to force the hooked head 43 into the first securing part 23 and then the receptacle section 25. The plunger 40 is then locked with the securing mount 20.

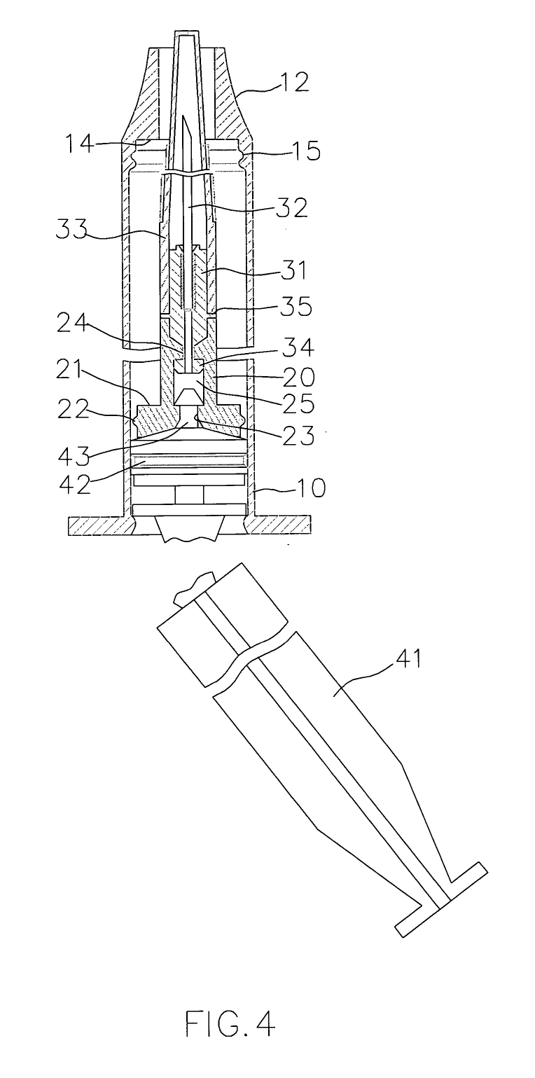

Referring to FIG. 4, when the plunger 40 is withdrawn from the barrel 10, it brings the securing mount and the needle unit thereon together to retract into the barrel 10. The plunger 40 is then broken to make sure that the needle will not expose out of the barrel again.

The inner wall of the through hole and the outer wall of the securing mount of the present invention are made of a single rubber material, so that the connection between the inner and the outer walls will be tight. The securing mount 20 can be manufactured by a usual mold and has an outer diameter slightly larger than the inner diameter of the through hole. Further, the flexibility of the rubber material making the mount is so used as to pass the through hole. Since the mount 20 and the hole are tightly engaged, the medical solution in the barrel 10 will not leak.

Specifically, the inner wall and outer wall off the securing mount 20 are made of a single rubber material, which facilitates the insertion of the needle unit 30 into the securing mount 20 and makes the contact between the outer wall and the through hole within the securing mount 20 tight. Since inner wall has a diameter slightly smaller than the outer diameter of the needle mount, the insertion of the needle unit 30 into the securing mount 20 is firm and stable.

Referring to FIG. 5, another preferred embodiment of the securing mount of the present invention will differ from that of the first preferred embodiment in that, as the securing mount is pulled back into the barrel, it will be tilted against the inner wall of the barrel. Therefore, the needle unit will not expose to the exterior.

More specifically, the securing mount 20a has a skewed shoulder portion 26a with a predetermined slope located between a first securing part 23a and a receptacle 25a, whereby the inner diameter of the first securing part 23a is aligned with an imaginary skewed axial line L so as to form a skewed through hole A.

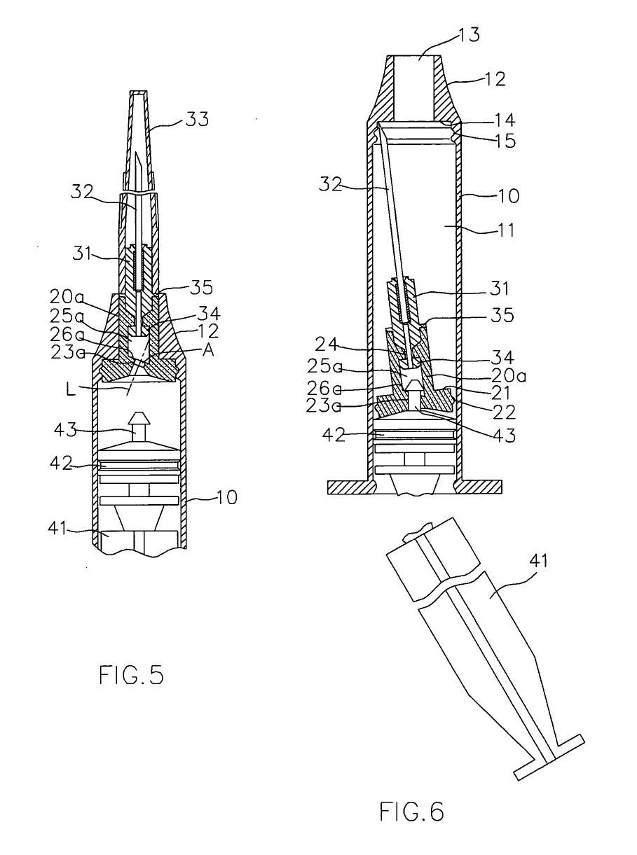

Referring to FIG. 6, when the hooked head 43 atop the plunger 40 is connected with the first securing part of the securing mount, one side of the inner wall will be pushed outwardly by the hooked head 43. Thereby, as the plunger is pulled back together with the securing mount 20a from the though hole 13 and retreated into the receptacle 11 in the barrel 10, the one-side pressure will be released, and the mount 20, now without support from both lateral sides, will be tilted aside due to the flexibility of the rubber mount 20. As a consequence, the needle 32 will be blocked by the surface of the stopper 14, and therefore the needle unit 30 will not expose to the exterior again.

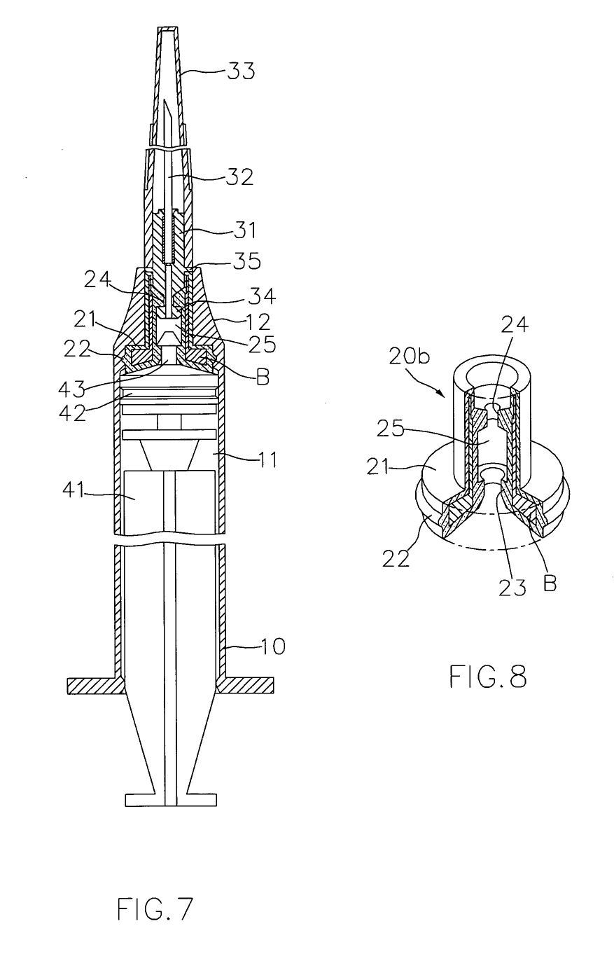

Referring to FIGS. 7 and 8, the second preferred embodiment of the present invention has a structure of similar the first preferred embodiment, and they differ in the following aspects.

1. The inner and outer walls of the securing mount of the first preferred embodiment are made of rubber material and by injection molding, which are short of toughness. Whereas the second preferred embodiment utilizes a securing mount of higher rigidity. The inner wall and the outer wall of the securing mount 20 are made of the same rubber material and by injection molding but provided with inner partitions to each house a reinforced layer of hard plastic material B, whereby the rigidity of the securing mount 20 is enhanced. This is to satisfy the need of syringes with large amounts of injection fluid.

2. However, the second preferred embodiment of the present invention, wherein the plastic reinforced layer B is covered with the rubber inner and outer walls, may have a skewed inner through hole therein. Thereby, as the securing mount is pulled back, it will be tilted on one side, because of the rubber flexibility of the first securing part, the skewed through hole and the inner wall.

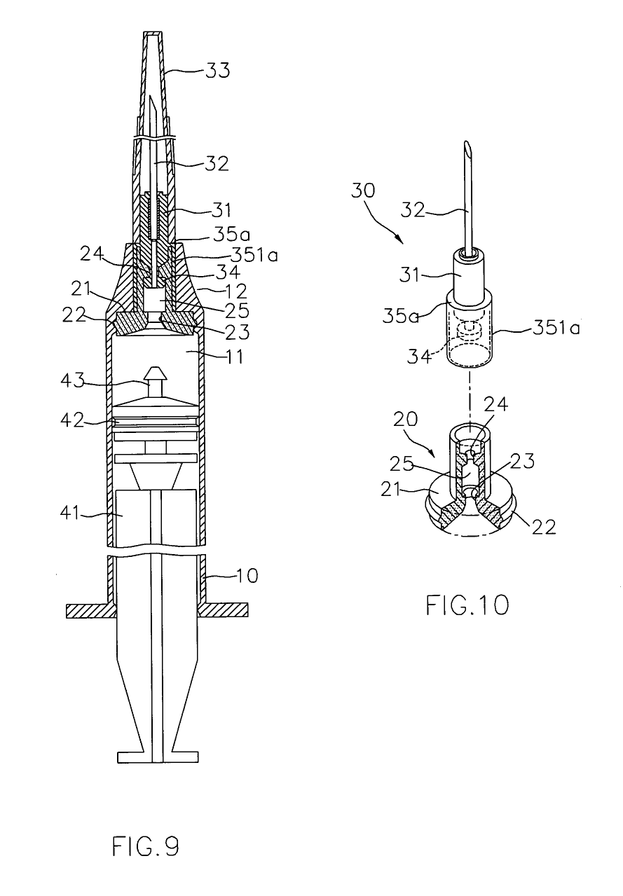

Referring to FIGS. 9 and 10, the third preferred embodiment of the present invention differs from the previous preferred embodiments in the local structure of the securing mount and the needle unit. More specifically, the differences are as follows.

1. The stop ring 35 for retaining the cover 33 around the needle mount 31 is a stepped flange. The cover 33 is a vertically extended hollow tube inserted on the securing mount 20, whereby the connecting portion 34 of the needle mount 31 is locked with the second securing part 24.

2. The cover 33, being a vertically extended hollow tube from the stop ring 35 can be made of a hard plastic material, whereby the securing mount 20 will be covered by a rigid cover and the insertion of the mount through the through hole will be smoother.

The present invention is thus described, and it will be obvious that the same may be varied in many ways. For example, the securing mount 20 and the needle mount 31 are injection-molded together; or the materials respectively for the securing mount 20 and the needle mount 31 can be locked together and then injection-molded together. Such variations are not to be regarded as a departure from the spirit and scope of the present invention, and all such modifications as would be obvious to one skilled in the art are intended to be included within the scope of the following claims.

What is claimed is:

1. A safety syringe, comprising:
a barrel being a hollow cylinder and having a tapered section at a front end thereof, said tapered section further including a longitudinal through hole of smaller diameter than a inner diameter of the inner receptacle defined by said barrel, a stopper being formed between said receptacle and said through hole;
a securing mount, made of a soft rubber material, and further including an inner wall and a outer wall located within said through hole of said tapered section, said securing mount further having a first securing part extended inwardly from said inner wall close to said receptacle and an enlarged disc portion, whereby said stopper of said barrel will retain said securing mount;
a needle unit situating at a free end opposite to said retained end where said first securing part is formed, said needle unit further including a needle mount and a needle inserted in said needle mount; and
a plunger inserted along said barrel and capable of being slid within said barrel, said plunger further including a push rod and a piston mounted on said push rod, said piston being provided with a hooked head that can be engaged with said first securing part as sad plunger is pushed forward deeply.

2. The safety syringe of claim 1 wherein said disc portion of said securing mount is provided with an annular projection, and wherein said barrel is provided a corresponding annular groove; thereby, said annular projection and said annular groove can be engaged.

3. The safety syringe of claim 1 wherein said securing mount has a second securing part on said inner wall far from said receptacle, whereby said first and second securing parts will define a receptacle section, and wherein a bottom end of said needle mount has a connecting portion, whereby said needle mount will be retained by said second securing part.

4. The safety syringe of claim 1 wherein said first securing part is provided with a through hole extended longitudinally along said securing mount.

5. The safety syringe of claim 1 wherein said first securing part is provided with a through hole that is skewed with respect to an imaginary axial line of said securing mount.

6. The safety syringe of claim 1 wherein said plastic needle mount is provided with an outwardly extending annular projection.

7. The safety syringe of claim 1 wherein said plastic needle mount is provided with a horizontally extending stepped flange and a hollow tubular cap extended vertically downward; thereby, said plastic needle mount can be mounted onto rubber securing mount.

8. The safety syringe of claim 1 wherein said rubber securing mount has an inner partition housing a plastic reinforced layer; said plastic reinforced layer is installed when said securing mount is formed by injection molding.
